(19) United States
(12) Patent Application Publication
(10) Pub. No.: US 2025/0325692 A1
(43) Pub. Date: Oct. 23, 2025

(54) METHODS OF TREATMENT USING ANTI-SEZ6 ANTIBODY DRUG CONJUGATES

(71) Applicant: AbbVie Inc., North Chicago, IL (US)

(72) Inventors: Randy R. Robinson, Mountain View, CA (US); Pooja Hingorani, Scottsdale, AZ (US); Edwin Jeng, San Francisco, CA (US); Jiuhong Zha, Chicago, IL (US); Dolonchampa Maji, San Mateo, CA (US); Kevin M.J. Huang, Worthington, OH (US); Laura M. Kreckler, Grayslake, IL (US)

(21) Appl. No.: 19/184,983

(22) Filed: Apr. 21, 2025

Related U.S. Application Data
(60) Provisional application No. 63/637,291, filed on Apr. 22, 2024.

(51) Int. Cl. A61K 47/68; A61P 35/00; C07K 16/18

(52) U.S. Cl. CPC A61K 47/68037; A61K 47/6857; A61P 35/00; C07K 16/18

(57) ABSTRACT

The present application pertains to, among other things, improved methods of treating advanced solid tumors, including small cell lung cancer (“SCLC”), relapsed/refractory (R/R) SCLC, high-grade central nervous system (“CNS”) tumors (glioblastoma (“GBM”), IDH-wild-type Grade 4; oligodendroglioma, IDH-mutant, and 1p/19q-codeleted Grade 3; astrocytoma, IDH-mutant Grade 3 or Grade 4), and high-grade neuroendocrine carcinomas/neuroendocrine tumors (“NECs/NETs”), using an anti-SEZ6 antibody drug conjugate (“anti-SEZ6 ADC”). In specific embodiments, the anti-SEZ6 ADC consists of a SEZ6-targeting antibody conjugated to a potent topoisomerase 1 inhibitor (Top1i) payload.

### 1. CROSS REFERENCE TO RELATED APPLICATION

This application claims the benefit under 35 U.S.C. § 119(e) of U.S. provisional application No. 63/637,291, filed on Apr. 22, 2024, the disclosure of which is incorporated herein by reference in its entirety.

### 2. SEQUENCE LISTING

The instant application contains a Sequence Listing which has been submitted electronically in XML format and is hereby incorporated by reference in its entirety. Said XML copy, created Apr. 17, 2024, is named SeqList_350794-66440v2.xml and is 21,161 bytes in size.

### 3. TECHNICAL FIELD

The present application pertains to, among other things, improved methods of treating solid tumors, including relapsed/refractory (R/R) small cell lung cancer (SCLC) tumors, high-grade central nervous system (CNS) tumors (glioblastoma (GEM), IDH (isocitrate dehydrogenase)-wild-type Grade 4; oligodendroglioma, IDH-mutant, and 1p/19q-codeleted Grade 3; astrocytoma, IDH-mutant Grade 3 or Grade 4), and high-grade neuroendocrine carcinomas (NECs)/neuroendocrine tumors (NETs)), by administering to a human subject having said solid tumor a therapeutically effective amount of an anti-SEZ6 antibody drug conjugate (“anti-SEZ6 ADC”). In specific embodiments, the anti-SEZ6 ADC consists of a SEZ6-targeting antibody conjugated to a potent topoisomerase 1 inhibitor (Top1i) payload.

### 4. BACKGROUND

Seizure-related homolog 6 (SEZ6) is a transmembrane protein found on the cell surface of select neuronal lineage cells (Shimizu-Nishikawa K, et al. Cloning and characterization of seizure-related gene, SEZ6. Biochem Biophys Res Commun. 1995; 216(1):382-9; Gunnersen J M, et al. SEZ6 is selectively expressed on neuroendocrine tumors, including small cell lung cancer (SCLC); Kudoh S, et al. Significance of achaete-scute complex homologue 1 (ASCL1) in pulmonary neuroendocrine carcinomas; RNA sequence analyses using small cell lung cancer cells and ASCL1-induced pulmonary neuroendocrine carcinoma cells. Histochem Cell Biol. 2020; 153(6):443-456). Small Cell Lung Cancer (SCLC) is an aggressive neuroendocrine carcinoma that represents 13-15% of all diagnosed lung cancers, with ˜260,000 new cases diagnosed annually worldwide with ˜33,000 diagnosed in the US leading to 25,000 deaths. The 5-year survival rate for extensive disease is 2% (˜65% of patients), and the limited stage disease 5-year survival rate is 15%.

Conventional therapeutic treatments for solid tumors are often ineffective, thus there remains a need to develop more targeted and potent therapies. An antibody drug conjugate (ADC) comprising an antibody targeting SEZ6 conjugated to a cytotoxic drug via a chemical linker provides a targeted therapy for treating patients with solid tumors.

There remains a need in the art for developing therapeutic methods using ADCs to treat solid tumors, in particular to find therapeutically effective and safe doses and dosing regimens of ADCs.

### 5. SUMMARY

Provided herein are methods of treating a solid tumor, including small cell lung cancer (SCLC) tumors, relapsed/refractory (R/R) SCLC tumors, high-grade central nervous system (CNS) tumors (glioblastoma (GBM), IDH-wild-type Grade 4; oligodendroglioma, IDH-mutant, and 1p/19q-codeleted Grade 3; astrocytoma, IDH-mutant Grade 3 or Grade 4), and high-grade neuroendocrine carcinomas (NECs)/neuroendocrine tumors (NETs)), by administering to a human subject having said solid tumor a therapeutically effective amount of an anti-SEZ6 ADC. using an anti-SEZ6 antibody drug conjugate (“anti-SEZ6 ADC”). In specific embodiments, the anti-SEZ6 ADC consists of a SEZ6-targeting antibody conjugated to a potent topoisomerase 1 inhibitor (Top1i) payload.

### 5.1 Illustrative Embodiments

The present disclosure includes the following non-limiting illustrative embodiments.
1. A method of treating a small cell lung cancer (“SCLC”) tumor in a human subject in need thereof or population of human subjects in need thereof, comprising administering intravenously every three weeks to the human subject having said SCLC tumor a therapeutically effective amount of 1.3 mg/kg, 1.5 mg/kg, 1.8 mg/kg, 2.5 mg/kg, 3.0 mg/kg, or 3.5 mg/kg of an anti-SEZ6 antibody drug conjugate (“anti-SEZ6 ADC”) having the following structure:

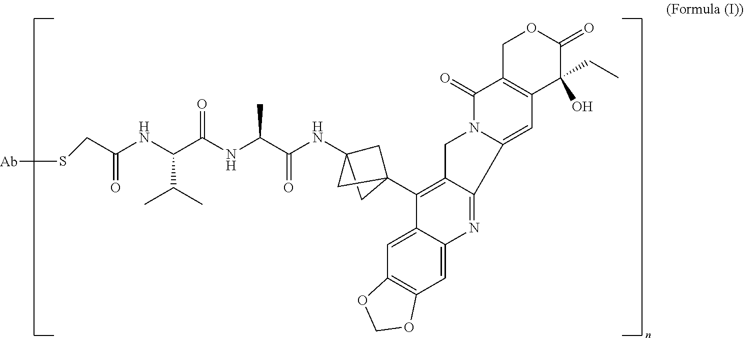

wherein n is an integer from 1 to 10, and wherein Ab is an anti-SEZ6 antibody comprising: two heavy chains each having an amino acid sequence shown as SEQ ID NO: 9 and two light chains each having an amino acid sequence shown as SEQ ID NO: 10, thereby treating said SCLC tumor.
2. The method of embodiment 1, wherein the SCLC tumor is refractory or relapsed.
3. The method of embodiment 1, wherein the SCLC tumor is previously untreated.
4. The method of embodiment 1, wherein the SCLC tumor is an extensive stage SCLC.
5. The method of any one of embodiments 1-3, wherein the SCLC tumor has progressed following at least one prior platinum-containing chemotherapy.
6. The method of any one of embodiments 1-3 wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.
7. The method of any one of embodiments 1-5, wherein administration of the anti-SEZ6 ADC achieves a partial response (PR) in the human subject.
8. The method of any one of embodiments 1-5, wherein administration of the anti-SEZ6 ADC achieves a complete response (CR) in the human subject.
9. The method of any one of embodiments 1-5, wherein administration of the anti-SEZ6 ADC achieves stable disease (SD) in the human subject.
10. The method of any one of embodiments 1-8, wherein the anti-SEZ6 ADC has an average drug-antibody ratio (DAR) of 6 or about 6.
11. The method of embodiment 1, wherein the SCLC tumor is a refractory or relapsed SCLC tumor, wherein a therapeutically effective amount of about 1.8 mg/kg to about 3.0 mg/kg of the anti-SEZ6 ADC is administered intravenously every three weeks to the human subject, wherein the anti-SEZ6 ADC has an average DAR of about 6.
12. The method of embodiment 1, wherein the SCLC tumor is a refractory or relapsed SCLC tumor, wherein a therapeutically effective amount of 1.8 mg/kg or 2.5 mg/kg of the anti-SEZ6 ADC is administered intravenously every three weeks to the human subject, wherein the anti-SEZ6 ADC has an average DAR of about 6.
13. The method of embodiment 1, wherein the SCLC tumor is a refractory or relapsed SCLC tumor, wherein a therapeutically effective amount of 1.8 mg/kg of the anti-SEZ6 ADC is administered intravenously every three weeks to the human subject, wherein the anti-SEZ6 ADC has an average DAR of about 6.
14. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6.
15. The method of embodiment 1, wherein the SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6
16. The method of embodiment 1, wherein the SCLC tumor is a refractory or relapsed SCLC tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves SD in the human subject.
17. The method of embodiment 1, wherein the SCLC tumor is a refractory or relapsed SCLC tumor, wherein a therapeutically effective amount of 1.8 mg/kg or 2.5 mg/kg of the anti-SEZ6 ADC is administered intravenously every three weeks to the human subject, wherein the anti-SEZ6 ADC has an average DAR of about 6, and wherein administration of the anti-SEZ6 ADC achieves SD in the human subject.
18. The method of embodiment 1, wherein the SCLC tumor is a refractory or relapsed SCLC tumor, wherein a therapeutically effective amount of 1.8 mg/kg of the anti-SEZ6 ADC is administered intravenously every three weeks to the human subject, wherein the anti-SEZ6 ADC has an average DAR of about 6, and wherein administration of the anti-SEZ6 ADC achieves SD in the human subject.
19. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC achieves SD in the human subject.
20. The method of embodiment 1, wherein SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC achieves SD in the human subject.
21. The method of embodiment 1, wherein the SCLC tumor is a refractory or relapsed SCLC tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
22. The method of embodiment 1, wherein the SCLC tumor is a refractory or relapsed SCLC tumor, wherein a therapeutically effective amount of 1.8 mg/kg, 2.5 mg/kg, or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
23. The method of embodiment 1, wherein the SCLC tumor is a refractory or relapsed SCLC tumor, wherein a therapeutically effective amount of 1.8 mg/kg or 2.5 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
24. The method of embodiment 1, wherein the SCLC tumor is a refractory or relapsed SCLC tumor, wherein a therapeutically effective amount of 1.8 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
25. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC achieves PR in the human subject.
26. The method of embodiment 1, wherein SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC achieves PR in the human subject.
27. The method of embodiment 1, wherein the SCLC tumor is a refractory or relapsed SCLC tumor, wherein a therapeutically effective amount of 1.8 mg/kg to 3.0 mg/kg of the anti-SEZ6 ADC is administered intravenously every three weeks to the human subject, wherein the anti-SEZ6ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.
28. The method of embodiment 1, wherein the SCLC tumor is a refractory or relapsed SCLC tumor, wherein a therapeutically effective amount of 1.8 mg/kg, 2.5 mg/kg, or 3.0 mg/kg of the anti-SEZ6 ADC is administered intravenously every three weeks to the human subject, wherein the anti-SEZ6ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.
29. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%,
30. The method of embodiment 1, wherein SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%,
31. The method of embodiment 1, wherein the SCLC tumor is a refractory or relapsed SCLC tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
32. The method of embodiment 1, wherein the SCLC tumor is a refractory or relapsed SCLC tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
33. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
34. The method of embodiment 1, wherein SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
35. The method of embodiment 1, wherein the SCLC tumor is a refractory or relapsed SCLC tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
36. The method of embodiment 1, wherein the SCLC tumor is a refractory or relapsed SCLC tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
37. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
38. The method of embodiment 1, wherein SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
39. The method of embodiment 1, wherein the SCLC tumor is a refractory or relapsed SCLC tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
40. The method of embodiment 1, wherein the SCLC tumor is a refractory or relapsed SCLC tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
41. The method of embodiment 1, wherein SCLC tumor is a previously untreated SCLC tumor, wherein a therapeutically effective amount of 1.5 mg/kg to 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
42. The method of embodiment 1, wherein SCLC tumor is a previously untreated SCLC tumor, wherein a therapeutically effective amount of 1.8 mg/kg of the anti-SEZ6 ADC is administered intravenously every three weeks to the human subject in combination with an anti-PD-L1 antibody, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
43. A method of treating a central nervous system (“CNS”) tumor in a human subject in need thereof or population of human subjects in need thereof, comprising administering intravenously every three weeks to the human subject having said CNS tumor a therapeutically effective amount of 1.3 mg/kg, 1.8 mg/kg, 2.5 mg/kg, 3.0 mg/kg, or 3.5 mg/kg of an anti-SEZ6 antibody drug conjugate (“anti-SEZ6 ADC”) having the following structure:

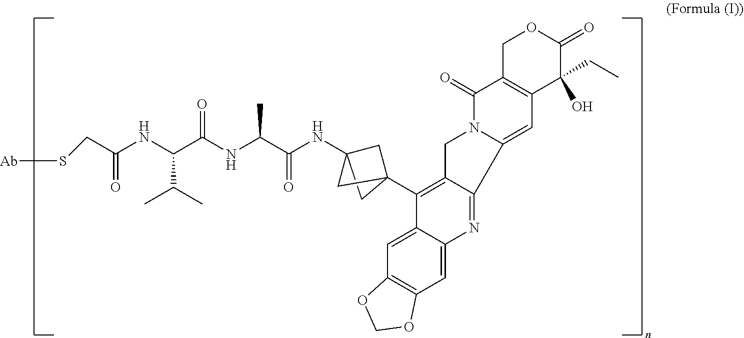

wherein n is an integer from 1 to 10, and wherein Ab is an anti-SEZ6 antibody comprising: two heavy chains each having an amino acid sequence shown as SEQ ID NO: 9 and two light chains each having an amino acid sequence shown as SEQ ID NO: 10, thereby treating said CNS tumor.
44. The method of embodiment 43, wherein the CNS tumor is refractory or relapsed.
45. The method of any one of embodiments 43-44, wherein the CNS tumor is an advanced solid tumor that has progressed on standard of care therapy and is not amenable to surgical resection.
46. The method of any one of embodiments 43-45, wherein the CNS tumor is glioblastoma (“GBM”), IDH-wildtype Grade 4; oligodendroglioma, IDH-mutant, and 1p/19q-codeleted Grade 3; astrocytoma, IDH-mutant Grade 3 or Grade 4.
47. The method of any one of embodiments 43-46, wherein the CNS tumor has progressed after treatment with at least prior radiation, and temozolomide or procarbazine, lomustine, and vincristine (PCV).
48. The method of any one of embodiments 43-47, wherein administration of the anti-SEZ6 ADC provides an overall response rate that is greater than 25%, greater than 30%, greater than 35%, greater than 40%, greater than 45%, greater than 50%, greater than 55%, greater than 60%, greater than 65%, greater than 70%, greater than 75%, or greater than 80%.
49. The method of any one of embodiments 43-48, wherein administration of the anti-SEZ6 ADC achieves a partial response (PR) in the human subject.
50. The method of any one of embodiments 43-48, wherein administration of the anti-SEZ6 ADC achieves a complete response (CR) in the human subject.
51. The method of any one of embodiments 43-48, wherein administration of the anti-SEZ6 ADC achieves stable disease (SD) in the human subject.
52. The method of any one of embodiments 43-48, wherein the anti-SEZ6 ADC has an average drug-antibody ratio (DAR) of 6 or about 6.
53. The method of embodiment 43, wherein the CNS tumor is a refractory or relapsed CNS tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6.
54. The method of embodiment 43, wherein the CNS tumor is a refractory or relapsed CNS tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
55. The method of embodiment 43, wherein the CNS tumor is a refractory or relapsed CNS tumor, wherein a therapeutically effective amount of 1.8 mg/kg, 2.5 mg/kg, or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
56. The method of embodiment 43, wherein the CNS tumor is a refractory or relapsed CNS tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.
57. The method of embodiment 43, wherein the CNS tumor is a refractory or relapsed CNS tumor, wherein a therapeutically effective amount of 1.8 mg/kg, 2.5 mg/kg, or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.
58. The method of claim 43, wherein the CNS tumor is a refractory or relapsed CNS tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
59. The method of claim 43, wherein the CNS tumor is a refractory or relapsed CNS tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
60. The method of claim 43, wherein the CNS tumor is a refractory or relapsed CNS tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
61. The method of claim 43, wherein the CNS tumor is a refractory or relapsed CNS tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
62. The method of claim 43, wherein the CNS tumor is a refractory or relapsed CNS tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
63. The method of claim 43, wherein the CNS tumor is a refractory or relapsed CNS tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
64. A method of treating a high-grade neuroendocrine carcinoma/neuroendocrine tumor (“NEC/NET”) in a human subject in need thereof or population of human subjects in need thereof, comprising administering intravenously every three weeks to the human subject having said NEC/NET tumor a therapeutically effective amount of 1.3 mg/kg, 1.8 mg/kg, 2.5 mg/kg, 3.0 mg/kg, or 3.5 mg/kg of an anti-SEZ6 antibody drug conjugate (“anti-SEZ6 ADC”) having the following structure:

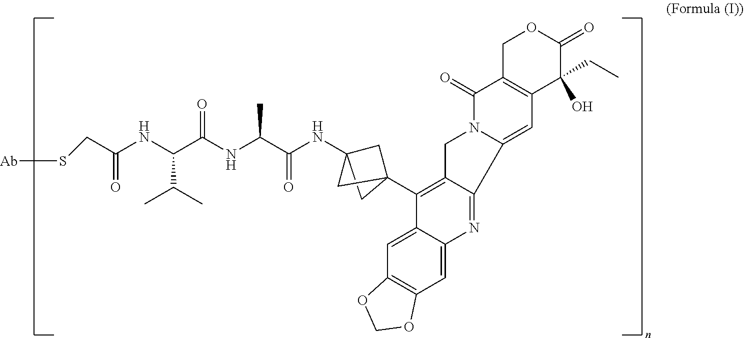

wherein n is an integer from 1 to 10, and wherein Ab is an anti-SEZ6 antibody comprising: two heavy chains each having an amino acid sequence shown as SEQ ID NO: 9 and two light chains each having an amino acid sequence shown as SEQ ID NO: 10, thereby treating said NEC/NET tumor.
65. The method of embodiment 64, wherein the NEC/NET tumor is refractory or relapsed.
66. The method of any one of embodiments 64-65, wherein the NEC/NET tumor is an advanced solid tumor that has progressed on standard of care therapy and is not amenable to surgical resection.
67. The method of any one of embodiments 64-66, wherein the NEC/NET tumor has progressed after treatment with at least a prior line of standard of care systemic chemotherapy.
68. The method of any one of embodiments 64-67, wherein the NEC/NET tumor has progressed after treatment with at least a tyrosine kinase inhibitor (TKI) or a rearranged during transfection (RET) inhibitor.
69. The method of any one of embodiments 64-68, wherein administration of the anti-SEZ6 ADC provides an overall response rate that is greater than 25%, greater than 30%, greater than 35%, greater than 40%, greater than 45%, greater than 50%, greater than 55%, greater than 60%, greater than 65%, greater than 70%, greater than 75%, or greater than 80%.
70. The method of any one of embodiments 64-68, wherein administration of the anti-SEZ6 ADC achieves a partial response (PR) in the human subject.
71. The method of any one of embodiments 64-68, wherein administration of the anti-SEZ6 ADC achieves a complete response (CR) in the human subject.
72. The method of any one of embodiments 64-68, wherein administration of the anti-SEZ6 ADC achieves stable disease (SD) in the human subject.
73. The method of any one of embodiments 64-72, wherein the anti-SEZ6 ADC has an average drug-antibody ratio (DAR) of 6 or about 6.
74. The method of embodiment 64, wherein the NEC/NET tumor is a refractory or relapsed NEC/NET tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6.
75. The method of embodiment 64, wherein the NEC/NET tumor is a refractory or relapsed NEC/NET tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
76. The method of embodiment 64, wherein the NEC/NET tumor is a refractory or relapsed NEC/NET tumor, wherein a therapeutically effective amount of 1.8 mg/kg, 2.5 mg/kg, or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC achieves PR in the human subject.
77. The method of embodiment 64, wherein the NEC/NET tumor is a refractory or relapsed NEC/NET tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.
78. The method of embodiment 64, wherein the NEC/NET tumor is a refractory or relapsed NEC/NET tumor, wherein a therapeutically effective amount of 1.8 mg/kg, 2.5 mg/kg, or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall response rate that is greater than 25%, greater than 30%, greater than 35%, greater than 40%, greater than 45%, greater than 50%, greater than 55%, greater than 60%, greater than 65%, greater than 70%, greater than 75%, or greater than 80%.
79. The method of embodiment 64, wherein the NEC/NET tumor is a refractory or relapsed NEC/NET tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
80. The method of embodiment 64, wherein the NEC/NET tumor is a refractory or relapsed NEC/NET tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides progression free survival (PFS) of at least 4 months, 4.5 months, 5 months, 5.5 months, 6 months, 7 months, 8 months or longer.
81. The method of embodiment 64, wherein the NEC/NET tumor is a refractory or relapsed NEC/NET tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
82. The method of embodiment 64, wherein the NEC/NET tumor is a refractory or relapsed NEC/NET tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides an overall survival (OS) of at least 8 months, 9 months, 10 months, 11 months, 12 months, 13 months, 14 months, 15 months, 16 months, or longer.
83. The method of embodiment 64, wherein the NEC/NET tumor is a refractory or relapsed NEC/NET tumor, wherein a therapeutically effective amount of 1.8 mg/kg to 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.
84. The method of embodiment 64, wherein the NEC/NET tumor is a refractory or relapsed NEC/NET tumor, wherein a therapeutically effective amount of 1.8 mg/kg, 2.5 mg/kg or 3.0 mg/kg of the anti-SEZ6 ADC is administered intravenously every three weeks to the human subject, wherein the anti-SEZ6 ADC has an average DAR of about 6, and wherein administration of the anti-SEZ6 ADC provides a duration of response (DoR) of at least at least 4 months, 5 months, 6 months, 7 months, 8 months, 9 months, 10 months, 11 months, 12 months, or longer.

### 6. BRIEF DESCRIPTION OF THE FIGURES

FIG. 1 is a schematic of the dose escalation scheme of the ongoing Phase 1 study of the ADC-1 composition. DL=dose level; BF=backfill; and BOIN=Bayesian optimal interval

### 7. DETAILED DESCRIPTION

Provided herein are methods of treating a solid tumor, including a small cell lung cancer (SCLC) tumor, relapsed/refractory (R/R) SCLC tumor and a high-grade central nervous system (CNS) tumor (glioblastoma (GBM), IDH-wild-type Grade 4; oligodendroglioma, IDH-mutant, and 1p/19q-codeleted Grade 3; astrocytoma, IDH-mutant Grade 3 or Grade 4, and high-grade neuroendocrine carcinomas (NECs)/neuroendocrine tumors (NETs)), by administering to a human subject having said solid tumor a therapeutically effective amount of an anti-SEZ6 ADC. A therapeutically effective amount of an anti-SEZ6 ADC is an amount sufficient to result in stable disease (SD), partial response (PR), or a complete response (CR) in the subject per RECIST v1.1 criteria for non-CNS tumors, or Rano criteria for CNS tumors, and/or increase in overall survival (OS) or progression free survival (PFS). In preferred embodiments, the anti-SEZ6 ADC is ADC-1 (see Section 6.1). In such preferred embodiment, ADC-1 is administered once every three (3) weeks (Q3W) in 21-day cycles at a dose of 1.3 mg/kg, 1.8 mg/kg, 2.5 mg/kg, 3.0 mg/kg, or 3.5 mg/kg.

### 7.1 Adc-1—an Anti-Sez6 Adc

In certain embodiments, an anti-SEZ-6 antibody drug conjugate of formula (I) is provided:

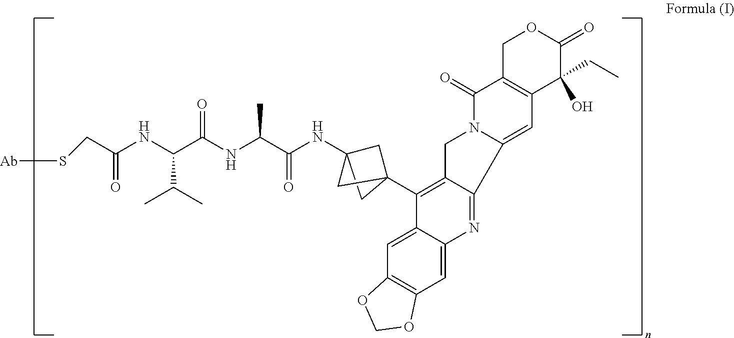

wherein antibody Ab is a humanized anti-SEZ6 IgG1 monoclonal antibody comprising two heavy chains each having an amino acid sequence shown as SEQ ID NO: 9 (variable region is bold and has an amino acid sequence shown as SEQ ID NO: 1; constant region is italicized; CDRs are underlined and have amino acid sequences shown as SEQ ID NOs: 3, 4 and 5 respectively, in order of appearance):

(SEQ ID NO: 9)
EVQLVQSGAEVKKPGESLKISCKGSGYSFTSSWINWVRQMPGKGLEWMGR
IYPGEGDTNYNGNFEGQVTISADKSISTAYLQWSSLKASDTAMYYCTRGL
VMDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSSDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG.

and two light chains, each having an amino acid sequence shown as SEQ ID NO: 10 (variable region is bold and has an amino acid sequence shown as SEQ ID NO: 2; constant region is italicized; CDRs are underlined and have amino acid sequences shown as SEQ ID NOs: 6, 7 and 8 respectively, in order of appearance):

(SEQ ID NO: 10)
EIVLTQSPATLSLSPGERATLSCRASQSVDYNGISYMHWYQQKPGQAPRL
LIYAASNVQSGIPARFSGSGSGTDFTLTISSLEPEDFAVYYCQQSIEDPP
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In embodiments, the heavy chains of the anti-SEZ-6 antibody drug conjugate comprise the amino acid sequence shown as SEQ ID NO: 9 with an additional C-terminal lysine.

In certain embodiments, conjugation of the linker-drug to the antibody is via a linkage formed with a sulfhydryl group of a cysteine residue of the antibody. In certain embodiments, n has a value of 1, 2, 3, 4, 5, 6, 7, 8, 9, or 10.

In certain embodiments, n is 6.

As used herein, “ADC-1” is an ADC composition comprising ADCs according to structural formula (I), wherein the Ab has two heavy chains each having an amino acid sequence shown as SEQ ID NO: 9 and two light chains each having an amino acid sequence shown as SEQ ID NO: 10 and a drug-antibody ratio (DAR) of about 6, with the predominant species having an n of 6. A procedure used to make ADC-1 is described in U.S. patent application Ser. No. 18/456,262. In an embodiment, a composition comprising a plurality of ADC-1 species with two or more different values of n is provided. In some embodiments, the composition of ADC-1 has an average drug-antibody ratio (DAR) of about 5.5, about 5.6, about 5.7, about 5.8, about 5.9, about 6.0, about 6.1, about 6.2, about 6.3, about 6.4, or about 6.5. In some embodiments, the composition of ADC-1 has an average of six molecules of the topoisomerase 1 inhibitor (Top1i) conjugated per anti-SEZ6 antibody. In some embodiments, the predominant species of ADC-1 in the composition has n=6. In some embodiments, ADC-1 has an average DAR of about 6.

### 7.2 Methods of Use

ADC-1 is unexpectedly more effective and safer than ABBV-011, a SEZ6-targeted, calicheamicin-based ADC. Even though ABBV-011 utilizes the same antibody sequences as ADC-1, ADC-1 has higher anti-tumor activity and reduced adverse events compared to ABBV-011.

ABBV-011 was administered at doses ranging from 0.3 to 2 mg/kg to subjects with SCLC tumors. The confirmed overall response rate (ORR) was 19% across all doses and 25% for those receiving 1 mg/kg. Grade 3 or higher treatment-emergent adverse effects (TEAEs) occurred in 65% of patients across all doses and 64% of patients treated at 1 mg/kg. Serious TEAEs were experienced by 45% of those who received ABBV-011 at 1 mg/kg. At the 1 mg/kg dose of ABV-011, 8% of patients discontinued treatment, 15% received a reduced dosage, and 30% interrupted treatment due to ABBV-011 related TEAEs.

In contrast, in the dose escalation arm of the First-in-Human trial, ADC-1 administered at doses higher than those used for ABBV-011, ranging from 1.3 to 3.5 mg/kg, resulted in a confirmed ORR of 43.8% across all doses. ADC-1 TEAEs of grade three or higher occurred in 77.4% of subjects across all doses, with 62.3% related to ADC-1 treatment. Serious TEAEs occurred in 32.1% of subjects across all doses, with 5.7% being related to ADC-1 treatment. Focusing on the 1.8 and 2.5 mg/kg dosing of ADC-1, serious TEAEs occurred in 33.3% of the subjects, with one (3.3%) serious TEAE related to ADC-1. 16.7% of subjects experienced a dose interruption related to ADC-1, and 16.7% of subjects experienced a dose reduction related to ADC-1.

In some embodiments, the ADC-1 composition is administered as monotherapy or in combination with an anti-PD-1 antibody (e.g., budigalimab), an anti-PD-L1 antibody, carboplatin, or cisplatin to treat a solid tumor (See sections 6.8.1-6.8.3 for examples of solid tumors). In one embodiment, the anti-PD-1 antibody binds PD-1 and is selected from the group consisting of pembrolizumab, nivolumab, cemiplimab, dostarlimab, and budigalimab. In one embodiment, the anti-PD-L1 antibody binds PD-L1 and is selected from the group consisting of atezolizumab, durvalumab, and avelumab.

Budigalimab is a recombinant anti-PD-1 (programmed cell death protein 1) immunoglobulin G1 monoclonal antibody. In some embodiments, budigalimab is administered intravenously once every three weeks at 375 mg. In some embodiments, budigalimab is administered approximately 30 minutes after completion of the ADC-1 infusion. Budigalimab, as used herein, refers to antibodies comprising heavy chains (HC) of SEQ ID NO:21 and light chains of SEQ ID NO:20 and products containing such antibodies wherein the antibodies or products have a name comprising the core name budigalimab with or without an FDA-designated suffix.

TABLE 2
Budigalimab sequences. CDRs are underlined.

| SEQ ID NO | Amino acid sequence (NC) |
|---|---|
| VH CDR1 | GYTFTHYGMN |
| SEQ ID NO: 11 | |
| VH CDR2 | WVNTYTGEPTYADDFKG |
| SEQ ID NO: 12 | |
| VH CDR3 | EGEGLGFGD |
| SEQ ID NO: 13 | |
| VL CDR1 | RSSQSIVHSHGDTYLE |
| SEQ ID NO: 14 | |
| VL CDR2 | KVSNRFS |
| SEQ ID NO: 15 | |
| VL CDR3 | FQGSHIPVT |
| SEQ ID NO: 16 | |
| VH | EIQLVQSGAEVKKPGSSVKVSCKASGYTFTHYGMNWVRQAPGQGLEWVGWVNTY |
| SEQ ID NO: 17 | TGEPTYADDFKGRLTFTLDTSTSTAYMELSSLRSEDTAVYYCTREGEGLGFGDW |
| | GQGTTVTVSS |
| VL | DVVMTQSPLSLPVTPGEPASISCRSSQSIVHSHGDTYLEWYLQKPGQSPQLLIY |
| SEQ ID NO: 18 | KVSNRFSGVPDRESGSGSGTDFTLKISRVEAEDVGVYYCFQGSHIPVTFGQGTK |
| | LEIKR |
| HC #1 | EIQLVQSGAEVKKPGSSVKVSCKASGYTFTHYGMNWVRQAPGQGLEWVGWVNTY |
| SEQ ID NO: 19 | TGEPTYADDFKGRLTFTLDTSTSTAYMELSSLRSEDTAVYYCTREGEGLGFGDW |
| | GQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKV |
| | EPKSCDKTHTCPPCPAPEAAGGPSVFLFPPKPKDTLMISRTPEVTCVVVDVSHE |
| | DPEVKFNWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSVMHEA |
| | LHNHYTQKSLSLSPGK |
| LC | DVVMTQSPLSLPVTPGEPASISCRSSQSIVHSHGDTYLEWYLQKPGQSPQLLIY |
| SEQ ID NO: 20 | KVSNRFSGVPDRESGSGSGTDFTLKISRVEAEDVGVYYCFQGSHIPVTFGQGTK |
| | LEIKRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSFNR |
| | GEC |
| HC #2 | EIQLVQSGAEVKKPGSSVKVSCKASGYTFTHYGMNWVRQAPGQGLEWVGWVNTY |
| SEQ ID NO: 21 | TGEPTYADDFKGRLTFTLDTSTSTAYMELSSLRSEDTAVYYCTREGEGLGFGDW |
| | GQGTTVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKV |
| | EPKSCDKTHTCPPCPAPEAAGGPSVFLFPPKPKDTLMISRTPEVTCVVVDVSHE |
| | DPEVKFNWYVDGVEVHNAKTKPREEQYNSTYRVVSVLTVLHQDWLNGKEYKCKV |
| | SNKALPAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCSVMHEA |
| | LHNHYTQKSLSLSPG |

In some embodiments, the anti-PD-1 antibody is budigalimab and is administered at a fixed IV dose at 375 mg once every 3 weeks (Q3W).

In some embodiments, the anti-PD-1 antibody is pembrolizumab and is administered at a flat dose of 200 mg once every 3 weeks (Q3W). In some embodiments, the anti-PD-1 antibody is pembrolizumab and is administered at a flat dose of 400 mg once every 6 weeks (Q6W). Doses and frequencies of administration of pembrolizumab are known in the art, for example, as specified in the KEYTRUDA® Prescribing Information.

In some embodiments, the anti-PD-1 antibody is nivolumab and is administered at a flat dose of 240 mg once every 2 weeks (Q2W). In some embodiments, the anti-PD-1 antibody is nivolumab and is administered at 3 mg/kg once every 2 weeks (Q2W). In some embodiments, the anti-PD-1 antibody is nivolumab and is administered at a flat dose of 360 mg once every 3 weeks (Q3W). In some embodiments, the anti-PD-1 antibody is nivolumab and is administered at a flat dose of 480 mg once every 4 weeks (Q4W). Doses and frequencies of administration of nivolumab are known in the art, for example, as specified in the OPDIVO® Prescribing Information.

In some embodiments, the anti-PD-1 antibody binds PD-L1, is atezolizumab, and is administered at a flat dose of 840 mg once every 2 weeks (Q2W). In some embodiments, the anti-PD-1 antibody binds PD-L1, is atezolizumab, and is atezolizumab and is administered at a flat dose of 1200 mg once every 3 weeks (Q3W). In some embodiments, the anti-PD-1 antibody binds PD-L1, is atezolizumab, and is administered at a flat dose of 1680 mg once every 4 weeks (Q4W). Doses and frequencies of administration of atezolizumab are known in the art, for example, as specified in the TECENTRIQ® Prescribing Information.

In some embodiments, carboplatin is administered intravenously once every three weeks at a dose calculated to achieve a target area under the curve of 5 mg/ml (AUC 5). In some embodiments, cisplatin is administered intravenously once every three weeks at 75 mg/m².

The term “a therapeutically effective amount” of an ADC used herein refers to an amount of an ADC sufficient to result in stable disease, partial response, or a complete response in the subject per RECIST v1.1, and/or increase in overall survival. RECIST v1.1 is published in New response evaluation criteria in solid tumors: Revised RECIST guideline (version 1.1), Eur Journal of Cancer 45 (2009) 228-247. “Complete Response (CR)” refers to disappearance of all target lesions. Any pathological lymph nodes (whether target or non-target) must have reduction in short axis to <10 mm. “Partial Response (PR)” refers to at least a 30% decrease in the sum of diameters of target lesions, taking as reference the baseline sum diameters.

### 7.2.1 Small Cell Lung Cancer (“SCLC”)

In one aspect, provided herein is a method of treating a small cell lung cancer (“SCLC”) tumor, comprising administering to a human subject having said SCLC tumor a therapeutically effective amount of ADC-1.

In embodiments, subjects with SCLC tumors are effectively treated with ADC-1 without screening for SEZ6 expression in the tumors. In an embodiment, subjects with SCLC tumors are treated without regard to the level of expression of SEZ6 in the SCLC tumor. In embodiments, subjects with an SCLC tumor are treated without determining the level of expression of SEZ6 in the SCLC tumor before, during or after treatment. In embodiments, the level of expression of SEZ6 in the tumor is not determined prior to administration of ADC-1. In embodiments, subjects respond favorably (i.e., PR, CR, SD) to ADC-1 treatment without screening for SCLC tumor SEZ6 expression.

In embodiments, ADC-1 is administered to the human patient once every three (3) weeks at a dose of 1.3 mg/kg, 1.5 mg/kg, 1.8 mg/kg, 2.5 mg/kg, 3.0 mg/kg, or 3.5 mg/kg. In a preferred embodiment, ADC-1 is administered intravenously. In a preferred embodiment, ADC-1 is administered once every three weeks until disease progression, withdrawal of consent, or unacceptable toxicity.

In certain embodiments, the SCLC tumor is at an advanced stage. In certain embodiments, the SCLC tumor is a relapsed SCLC tumor. In certain embodiments, the SCLC tumor is a relapsed or refractory SCLC tumor.

In embodiments, the SCLC is Extensive Stage SCLC (ES-SCLC).

In certain embodiments, the SCLC tumor has progressed following at least one prior platinum-containing chemotherapy.

In specific embodiments, the SCLC tumor is previously untreated.

In specific embodiments, a therapeutically effective amount of 1.8 mg/kg to 2.5 mg/kg, or about 1.8 mg/kg to about 2.5 mg/kg of the anti-SEZ6 ADC is administered intravenously every three weeks to the human subject. In specific embodiments, a therapeutically effective amount of 1.8 mg/kg of the anti-SEZ6 ADC is administered intravenously every three weeks to the human subject.

In specific embodiments, the SCLC tumor is advanced, a therapeutically effective amount of 1.8 mg/kg to 3.0 mg/kg, or about 1.8 mg/kg to about 3.0 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the SCLC tumor is advanced, a therapeutically effective amount of 1.8 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the SCLC tumor is advanced, a therapeutically effective amount of 2.5 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the SCLC tumor is advanced, a therapeutically effective amount of 3.0 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the SCLC tumor is previously untreated, a therapeutically effective amount of 1.5 mg/kg to 1.8 mg/kg, or about 1.5 mg/kg to about 1.8 mg/kg of the anti-SEZ6 ADC is administered intravenously every three weeks to the human subject in combination with an anti-PD-L1 antibody, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the SCLC tumor is previously untreated, a therapeutically effective amount of 1.8 mg/kg of the anti-SEZ6 ADC is administered intravenously every three weeks in combination with an anti-PD-L1 antibody to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In various embodiments, the SCLC tumor expresses SEZ6. In certain embodiments, the SCLC tumor overexpresses SEZ6.

In various embodiments, the expression level of SEZ6 of a SCLC tumor (e.g., whether the SCLC tumor overexpresses SEZ6 or expresses SEZ6) is assessed using an immunohistochemistry (IHC) assay or an assay measuring mRNA expression.

### 7.2.2 Central Nervous System (“CNS”) Tumors

In one aspect, provided herein is a method of treating a central nervous system (“CNS”) tumor, comprising administering to a human subject having said CNS tumor a therapeutically effective amount of ADC-1.

In embodiments, ADC-1 is administered to the human subject once every three (3) weeks at a dose of 1.3 mg/kg, 1.8 mg/kg, 2.5 mg/kg, 3.0 mg/kg, or 3.5 mg/kg. In a preferred embodiment, ADC-1 is administered intravenously. In a preferred embodiment, ADC-1 is administered once every three weeks until disease progression, withdrawal of consent, or unacceptable toxicity.

In certain embodiments, the CNS tumor is at an advanced stage. In certain embodiments, the CNS tumor is a relapsed CNS tumor. In certain embodiments, the CNS tumor is a relapsed or refractory CNS tumor.

In some embodiments, the CNS tumor is a high-grade CNS tumor such as GBM, IDH-wildtype Grade 4; oligodendroglioma, IDH-mutant, and 1p/19q-codeleted Grade 3; astrocytoma, IDH-mutant Grade 3 or Grade 4. In some embodiments, the CNS tumor was previously treated via surgical resection, received prior radiation, and temozolomide or procarbazine, lomustine, and vincristine (PCV).

In specific embodiments, a therapeutically effective amount of 1.8 mg/kg to 2.5 mg/kg, or about 1.8 mg/kg to about 3.0 mg/kg of the anti-SEZ6 ADC is administered intravenously every three weeks to the human subject.

In specific embodiments, the CNS tumor is advanced, a therapeutically effective amount of 1.8 mg/kg to 3.0 mg/kg, or about 1.8 mg/kg to about 3.0 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the CNS tumor is advanced, a therapeutically effective amount of 1.8 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the CNS tumor is advanced, a therapeutically effective amount of 2.5 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the CNS tumor is advanced, a therapeutically effective amount of 3.0 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In various embodiments, the CNS tumor expresses SEZ6. In certain embodiments, the CNS tumor overexpresses SEZ6.

In various embodiments, the expression level of SEZ6 of a CNS tumor (e.g., whether the CNS tumor overexpresses SEZ6 or expresses SEZ6) is assessed using an immunohistochemistry (IHC) assay or an assay measuring mRNA expression.

### 7.2.3 High-grade Neuroendocrine Carcinoma/Neuroendocrine Tumor (“NEC/NET”)

In one aspect, provided herein is a method of treating a high-grade neuroendocrine carcinoma/neuroendocrine (“NEC/NET”) tumor, comprising administering to a human subject having said NEC/NET tumor a therapeutically effective amount of ADC-1. Advantageously, a wide range of NEC/NET tumors respond favorably to treatment with ADC-1, resulting in stable disease or a partial response.

In an embodiment, ADC-1 is administered to the human patient once every three (3) weeks at a dose of 1.3 mg/kg, 1.8 mg/kg, 2.5 mg/kg, 3.0 mg/kg, or 3.5 mg/kg. In a preferred embodiment, ADC-1 is administered intravenously. In a preferred embodiment, ADC-1 is administered once every three weeks until disease progression, withdrawal of consent, or unacceptable toxicity.

In certain embodiments, the NEC/NET tumor is at an advanced stage. In certain embodiments, the NEC/NET tumor is a relapsed NEC/NET tumor. In certain embodiments, the NEC/NET tumor is a relapsed or refractory NEC/NET tumor.

In some embodiments, the NEC/NET tumor is a neuroendocrine prostate cancer (“NEPC”) tumor, a gastroenteropancreatic neuroendocrine carcinoma tumor (“GEP-NEC”), a large cell neuroendocrine carcinoma tumor (“LCNEC”), a SCLC tumor transformed from epidermal growth factor receptor (“EGFR”) mutant non-small cell lung cancer (“NSCLC”), a medullary thyroid cancer (“MTC”) tumor, or other NEC tumors or a Grade 3 NET tumor.

In some embodiments, the NEC/NET tumor was previously treated with at least one prior line of standard of care systemic chemotherapy (e.g., platinum+etoposide, platinum+irinotecan, FOLFOX, FOLFIRI, FOLFIRINOX, others).

In some embodiments, the NEC/NET tumor was previously treated with at least one prior line of standard of care therapy (e.g., tyrosine kinase inhibitor (TKI) or a rearranged during transfection (RET) inhibitor, if applicable).

In specific embodiments, a therapeutically effective amount of 1.8 mg/kg to 2.5 mg/kg, or about 1.8 mg/kg to about 3.0 mg/kg of the anti-SEZ6 ADC is administered intravenously every three weeks to the human subject.

In specific embodiments, the NEC/NET tumor is advanced, a therapeutically effective amount of 1.8 mg/kg to 3.0 mg/kg, or about 1.8 mg/kg to about 3.0 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the NEC/NET tumor is advanced, a therapeutically effective amount of 1.8 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the NEC/NET tumor is advanced, a therapeutically effective amount of 2.5 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In specific embodiments, the NEC/NET tumor is advanced, a therapeutically effective amount of 3.0 mg/kg of the anti-SEZ6 ADC is administered intravenously every three weeks to the human subject, the anti-SEZ6 ADC has an average DAR of about 6, and administration of the anti-SEZ6 ADC provides an overall response rate (ORR) that is greater than 25%, greater than 30%, greater than 35%, greater than 40%, greater than 45%, greater than 50%, greater than 55%, greater than 60%, greater than 65%, greater than 70%, greater than 75%, or greater than 80%.

In various embodiments, the NEC/NET tumor expresses SEZ6. In certain embodiments, the NEC/NET tumor overexpresses SEZ6.

In embodiments, the expression level of SEZ6 of a NEC/NET tumor (e.g., whether the NEC/NET tumor overexpresses SEZ6 or expresses SEZ6) is assessed using an immunohistochemistry (IHC) assay or an assay measuring mRNA expression.

### 7.2.4 Therapeutic Endpoints

In various aspects and embodiments of a treatment method described herein, administration of the anti-SEZ6 ADC (e.g., ADC-1) provides an overall response rate that is superior to that of the standard of care therapy(ies) for the tumor. In various aspects and embodiments of a treatment method described herein, administration of the anti-SEZ6 ADC (e.g., ADC-1) for treatment of SCLC, CNS and NEC/NET tumors provides an overall response rate that is greater than 20%, greater than, 25%, greater than 30%, greater than 35%, greater than 40%, greater than 45%, greater than 50%, greater than 55%, greater than 60%, greater than 65%, greater than 70%, greater than 75%, or greater than 80%.

In various aspects and embodiments of a treatment method described herein, administration of the anti-SEZ6 ADC (e.g., ADC-1) for treatment of SCLC, CNS and NEC/NET tumors exhibits an acceptable safety and tolerability profile.

In various aspects and embodiments of a treatment method described herein, administration of the anti-SEZ6 ADC (e.g., ADC-1) for treatment of SCLC, CNS and NEC/NET tumors achieves a partial response (PR) in a population of human subjects. In one embodiment, administration of the anti-SEZ6 ADC (e.g., ADC-1) achieves a partial response (PR) in at least 10% of the human subjects such as 10%, 15%, 20%, 25%, 30%, 35%, 40%, 45%, 50%, 55%, 60%, 65, 70%, 75%, 80%, 85%, 90%, 95%, 100%, or any value in-between.

In various aspects and embodiments of a treatment method described herein, administration of the anti-SEZ6 ADC (e.g., ADC-1) achieves a complete response (CR) in in a population of human subjects. In one embodiment, administration of the anti-SEZ6 ADC (e.g., ADC-1) for treatment of SCLC, CNS and NEC/NET tumors achieves a complete response (CR) in at least 10% of the human subjects such as 10%, 15%, 20%, 25%, 30%, 35%, 40%, 45%, 50%, 55%, 60%, 65, 70%, 75%, 80%, 85%, 90%, 95%, 100%, or any value in-between.

In various aspects and embodiments of a treatment method described herein, administration of the anti-SEZ6 ADC (e.g., ADC-1) achieves stable disease (SD) in a population of human subjects. In one embodiment, administration of the anti-SEZ6 ADC (e.g., ADC-1) for treatment of SCLC, CNS and NEC/NET tumors achieves stable disease (SD) in at least 10% of the human subjects such as 10%, 15%, 20%, 25%, 30%, 35%, 40%, 45%, 50%, 55%, 60%, 65, 70%, 75%, 80%, 85%, 90%, 95%, 100%, or any value in-between.

In various aspects and embodiments of a treatment method described herein, administration of the anti-SEZ6 ADC (e.g., ADC-1) for treatment of SCLC, CNS and NEC/NET tumors achieves a clinical benefit rate (CBR) of at least 50% in the human subject such as 50%, 55%, 60%, 65%, 70%, 75%, 80%, 85%, 90%, 95%, 100%, or any value in-between.

In various aspects and embodiments of a treatment method described herein, administration of the anti-SEZ6 ADC (e.g., ADC-1) for treatment of SCLC, CNS and NEC/NET tumors achieves progression-free survival (PFS) of at least 4 months in a population of human subjects such as 4, 4.5, 5, 5.5, 6, 7, or 8 months or greater, or any value in-between.

In various aspects and embodiments of a treatment method described herein, administration of the anti-SEZ6 ADC (e.g., ADC-1) for treatment of SCLC, CNS and NEC/NET tumors achieves an overall response rate (ORR) of at least 20% in a population of human subjects, such as 20%, 25%, 30%, 35%, 40%, 45%, 50%, 55%, 60%, 65, 70%, 75%, 80%, 85%, 90%, 95%, 100%, or any value in-between.

In various aspects and embodiments of a treatment method described herein, administration of the anti-SEZ6 ADC (e.g., ADC-1) for treatment of SCLC, CNS and NEC/NET tumors achieves a median duration of response (DoR) of at least 4 months, such as 4 months, 5 months, 6 months, 7 months, 8 months, 9 months, 10 months, 11 months, 12 months, or longer.

In various aspects and embodiments of a treatment method described herein, administration of the anti-SEZ6 ADC (e.g., ADC-1) for treatment of SCLC, CNS and NEC/NET tumors achieves a median overall survival (OS) of at least 8 months, 9 months, 10 months, 11 months, 12 months, 13 months, 14 months, 15 months, 16 months, or longer.

In various aspects and embodiments, one or more or all of the eligibility criteria listed in Section 7.1.1 are used to determine whether a human subject is eligible for treatment.

In one embodiment, the confirmed complete response/partial response (CR/PR) is assessed by investigators per Response Evaluation Criteria in Solid Tumors (RECIST), version 1.1 for extracranial solid tumors. In one embodiment, the confirmed complete response/partial response (CR/PR) is assessed by Response Assessment for Neuro-Oncology (RANO) for subjects with high-grade CNS tumors (GBM, IDH-wildtype Grade 4; oligodendroglioma, IDH-mutant, and 1p/19q-codeleted Grade 3; astrocytoma, IDH-mutant Grade 3 or Grade 4).

### 8. EXAMPLES

The following Examples, which highlight certain features and properties of the exemplary embodiments of the methods described herein are provided for purposes of illustration.

### 8.1 Example 1: Phase 1 Study of the ADC-1 Composition as Monotherapy and in Combination with Budigalimab (Anti-PD-1), Carboplatin, or Cisplatin in Adult Subjects with Advance Solid Tumors

Disclosed herein is an ongoing Phase 1 first-in-human dose escalation and dose expansion study aiming to evaluate the safety, pharmacokinetics (PK), and antitumor activity of an ADC-1 composition (SEQ ID NOs: 9 and 10; anti-SEZ6 antibody and Top1 inhibitor payload; formula (I), with a DAR of about 6)) as monotherapy and in combination with budigalimab (anti-PD-1), carboplatin, or cisplatin in adult subjects with advance solid tumors. Primary objectives of the study are: 1. to evaluate the safety, tolerability, and pharmacokinetics of the ADC-1 composition administered as monotherapy and in combination with either budigalimab, carboplatin or cisplatin; 2. to determine the recommended Phase 2 dose (RP2D) of the ADC-1 composition as monotherapy; and 3. to evaluate the antitumor activity of the ADC-1 composition in subjects with relapsed/refractory (R/R) small cell lung cancer (SCLC), high-grade central nervous system (CNS) tumors (glioblastoma (GBM), IDH-wild-type Grade 4; oligodendroglioma, IDH-mutant, and 1p/19q-codeleted Grade 3; astrocytoma, IDH-mutant Grade 3 or Grade 4, and high-grade neuroendocrine carcinomas (NECs)/neuroendocrine tumors (NETs)).

This ongoing study includes a monotherapy dose escalation (Part 1). During monotherapy dose determination and expansion in R/R SCLC (Part 2) the number of subjects enrolled depends on the number and size of the randomized dose determination cohorts and if there is a requirement of a SEZ6 expression threshold and/or line of therapy. Subjects in Part 2a are randomized evenly between at least 2 dose cohorts. For combination dose escalation and expansion with budigalimab or platinum chemotherapy (Parts 3a and 3b, respectively), up to 30 and 60 subjects are enrolled respectively and are sufficient for an initial safety dataset to enable future efficacy studies. For monotherapy dose expansion in R/R CNS tumors or high-grade NECs/NETs (Parts 4a and 4b, respectively) up to 40 subjects each are enrolled.

In the study, the ADC-1 composition is administered as an intravenous (IV) infusion once every 3 weeks (Q3Wk) in 21-days cycles until disease progression, withdrawal of consent or unacceptable toxicity. The ADC-1 composition is administered at a starting dose of 1.3 mg/kg with increasing dose levels that include 2.5 mg/kg, 3.0 mg/kg, and 3.5 mg/kg. Carboplatin is administered intravenously at AUC (area under the concentration-time curve) 5 and cisplatin is administered intravenously at 75 mg/m² on Day 1 of each 21-day cycle. Budigalimab is administered at a fixed IV dose at 375 mg Q3Wk. The duration of the study for each subject is approximately 3 years and includes a 14-day screening period followed by an approximately 1-year treatment period and up to 2-year follow-up after the last dose of study drug.

### 8.1.1 Key Eligibility Criteria

1. Age≥18 years.
2. Eastern Cooperative Oncology Group (ECOG) performance status of 0 or 1.
3. The following laboratory values criteria must be met within 7 days prior to the first dose of study drug:
   Alanine aminotransferase (ALT) and aspartate aminotransferase (AST)≤3.0×the upper limit of normal (ULN).
      For subjects with liver metastases: ALT and AST≤5.0×ULN
   Total bilirubin≤1.5×ULN. Subjects with documented Gilbert's syndrome may have a total bilirubin≤3×ULN).
   Creatinine clearance≥30 mL/min calculated using the Modification of Diet in Renal Disease (MDRD) equation.
   Absolute neutrophil count≥1,500/mm³ (with no granulocyte-colony stimulating factor [G-CSF] in the past 10 days).
   Platelet count≥100,000/μL (with no platelet transfusion in the past 7 days).
   Hemoglobin≥8 g/dL (with no red blood cell [RBC]transfusion in the past 7 days).
4. QT interval corrected for heart rate (QTc)≤450 msec (males) or ≤470 msec (females) using Fridericia's correction, and an ejection fraction of ≥50% as measured by echocardiogram or multigated acquisition (MUGA) scan at Screening.
5. Subjects must have one of the following histologically or cytologically confirmed advanced relapsed or refractory solid tumors:
6. Part 1 only: Solid tumors with potential SEZ6 expression including (but not limited to) SCLC, high-grade CNS tumors (gliboblastoma (“GBM”), IDH-wildtype Grade 4; oligodendroglioma, IDH-mutant, and 1p/19q-codeleted Grade 3; astrocytoma, IDH-mutant Grade 3 or Grade 4), high-grade NECs/NETs (e.g., Neuroendocrine prostate cancer (“NEPC”), GEP-NECs, large cell neuroendocrine carcinomas (“LCNECs”), SCLC transformed from epidermal growth factor receptor (“EGFR”) mutant non-small cell lung cancer (“NSCLC”), medullary thyroid cancer (“MTC”), and other NECs, or Grade 3 NETs (atypical lung carcinoids that have received prior chemotherapy are allowed))
7. Part 2 only: SCLC
8. Part 3a only: SCLC or high-grade NECs/NETs
9. Part 3b only: SCLC who have only progressed following a frontline regimen containing a platinum-based chemotherapy (i.e., second-line SCLC subjects) or high-grade NECs/NETs
10. Part 4a only: High-grade CNS tumors
11. Part 4b only: High-grade NECs/NETs
12. Subjects must have progressed on or after standard of care (SoC) therapy as follows and with no curative therapy available (subjects who have refused, are considered ineligible for, or are intolerant of SoC therapy are eligible):
   For SCLC, subjects must have relapsed or refractory disease following at least 1 prior platinum-containing chemotherapy. For the purposes of this study, a line of therapy is defined as ≥1 complete cycle of either a single agent or combination of drugs, including any planned sequential therapy of various regimens (e.g., 4 cycles of initial therapy with carboplatin-etoposide and atezolizumab or durvalumab followed by maintenance with atezolizumab or durvalumab is considered first line).
   For high-grade CNS tumors, subjects must have had prior surgical resection, received prior radiation, and temozolomide or procarbazine, lomustine, and vincristine (PCV).
   For high-grade NECs/NETs, subjects must have received at least one prior line of SoC systemic chemotherapy (e.g., platinum+etoposide, platinum+irinotecan, FOLFOX, FOLFIRI, FOLFIRINOX, others). For medullary thyroid cancer, subjects must have received at least one prior line SoC therapy (e.g., tyrosine kinase inhibitor (TKI) or a rearranged during transfection (RET) inhibitor, if applicable).
13. Measurable disease per RECIST version 1.1 for subjects with extracranial solid tumors or RANO for subjects with primary high-grade CNS tumors (GBM, IDH-wildtype Grade 4; oligodendroglioma, IDH-mutant, and 1p/19q-codeleted Grade 3; astrocytoma, IDH-mutant Grade 3 or Grade 4).
14. Primary CNS tumors within 12 weeks from radiation therapy should have unequivocal progression as documented by either tumor recurrence predominantly outside of radiation field on magnetic resonance imaging (MRI) or confirmed on tumor biopsy.
15. Subjects with brain metastases from an extracranial solid tumor are eligible if the brain metastases are:
   Previously treated, currently stable and not requiring steroids or anticonvulsants to control associated symptoms.
   Untreated, asymptomatic, with fewer than 5 lesions and less than 5 mm in size each and not requiring steroids or anticonvulsants to control associated symptoms.
16. Fresh or archival tumor tissue available for submission, for retrospective SEZ6 expression analysis, as per tissue submission guidelines in the Laboratory Manual.
   Subjects that have previously provided tissue sampling for SEZ6 testing in other AbbVie studies and meet the sampling requirements outlined by the Sponsor are not required to provide further tissue samples for this study.
17. No history of interstitial lung disease (ILD) or pneumonitis that required treatment with systemic steroids, nor any evidence of active ILD or pneumonitis.
18. No history of idiopathic pulmonary fibrosis or organizing pneumonia (e.g., bronchiolitis obliterans).
19. No prior treatment with an ADC that consists of a Top1 inhibitor payload.
20. Part 2 (SCLC monotherapy dose determination and expansion) only: No prior treatment with a SEZ6-targeted antibody drug conjugate.

### 8.1.2 Pharmacokinetic Analysis

Whole blood samples are collected into appropriately labeled tubes and processed. Serum concentrations of ADC-1 (total antibody and ADC), plasma concentrations of the unconjugated Top1 inhibitor payload, and relative titers of serum ADC-1 anti-drug antibody (ADA) are determined using validated methods.

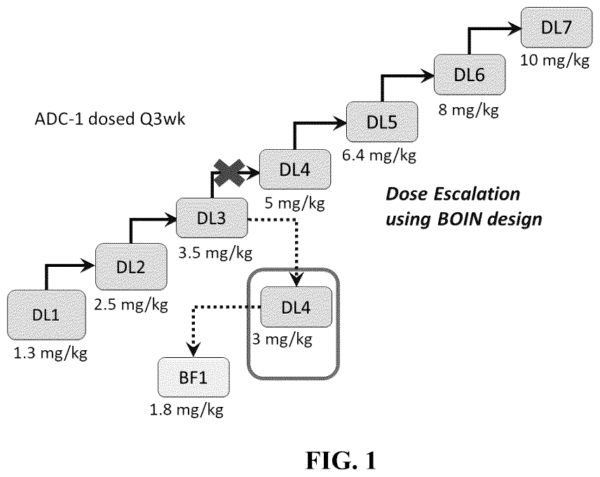

This Phase 1 study was initiated and is ongoing. The dose escalation scheme is shown in FIG. 1.

### 8.2 Example 2: Dose Escalation Results from a First-In-Human Study of ADC-1, a Novel SEZ6-Targeting Antibody-Drug Conjugate, in Advanced Solid Tumors

As of Mar. 20, 2024 data from 53 patients has been collected from Part 1 of the Phase 1 study described in Example 1. Specifically, data has been collected from 3 patients enrolled at 1.3 mg/kg, 15 patients enrolled at 1.8 m/g/kg, 15 patients enrolled at 2.5 mg/kg, 15 patients enrolled at 3.0 mg/kg, and 5 patients enrolled at 3.5 mg/kg of the ADC-1 composition. Median age was 64 years old (range, 32-81), 33 (62.3%) of the patients were male, and the tumor types included SCLC (n=23), NEN (neuroendocrine neoplasm) (n=25), and CNS (n=5). The median number of prior treatment lines was 3.0 (range, 1-6). For the SCLC tumors, the majority of patients received greater than 1 prior line of therapy and were platinum resistant to 1 line of therapy. For the NEN tumors, the most common were gastro-enteropancreatic in origin.

The ADC-1 composition described in Example 1 demonstrated a tolerable safety profile as shown in Table 3. The maximum tolerable dose (MTD) was 3.0 mg/kg Q3wk.

TABLE 3
Safety profile of the ADC-1 composition and all doses
collected from clinical study as of Mar. 20, 2024.
Summary of Treatment Emergent Adverse Events (TEAEs): ADC-1 Monotherapy

| | 1.3 mg/kg | | 1.8 mg/kg | | 2.5 mg/kg | | 3.0 mg/kg | | 3.5 mg/kg | | Total | |
|---|---|---|---|---|---|---|---|---|---|---|---|---|
| | (n = 3) | | (n = 15) | | (n = 15) | | (n = 15) | | (n = 5) | | (n = 53) | |
| Any grade | 3 | (100) | 14 | (93.3) | 15 | (100) | 15 | (100) | 5 | (100) | 52 | (98.1) |
| TEAEs | | | | | | | | | | | | |
| Related to | 3 | (100) | 14 | (93.3) | 14 | (93.3) | 15 | (100) | 5 | (100) | 51 | (96.2) |
| ADC-1 | | | | | | | | | | | | |
| Grade ≥3 | 2 | (66.7) | 8 | (53.3) | 12 | (80.0) | 14 | (93.3) | 5 | (100) | 41 | (77.4) |
| TEAEs | | | | | | | | | | | | |
| Related to | 2 | (66.7) | 3 | (20.0) | 10 | (66.7) | 13 | (86.7) | 5 | (100) | 33 | (62.3) |
| ADC-1 | | | | | | | | | | | | |
| Serious | 1 | (33.3) | 6 | (40.0) | 4 | (26.7) | 4 | (26.7) | 2 | (40) | 17 | (32.1) |
| Adverse | | | | | | | | | | | | |
| Events | | | | | | | | | | | | |
| Related to | 0 | (0) | 0 | (0) | 1 | (6.7) | 1 | (6.7) | 1 | (20) | 3 | (5.7) |
| ADC-1 | | | | | | | | | | | | |
| Dose | 0 | (0) | 0 | (0) | 0 | (0) | 1 | (7.7) | 1 | (20) | 2 | (4.1) |
| Limiting | | | | | | | | | | | | |
| Toxicities | | | | | | | | | | | | |
| TEAE | 1 | (33.3) | 6 | (40.0) | 7 | (46.7) | 11 | (73.3) | 4 | (80) | 29 | (54.7) |
| Leading to | | | | | | | | | | | | |
| Dose | | | | | | | | | | | | |
| Interruption | | | | | | | | | | | | |
| Related to | 1 | (33.3) | 1 | (6.7) | 4 | (26.7) | 7 | (46.7) | 3 | (60.0) | 16 | (30.2) |
| ADC-1 | | | | | | | | | | | | |
| TEAE | 1 | (33.3) | 4 | (26.7) | 2 | (13.3) | 10 | (66.7) | 4 | (80) | 21 | (39.6) |
| Leading to | | | | | | | | | | | | |
| Dose | | | | | | | | | | | | |
| Reduction | | | | | | | | | | | | |
| Related to | 1 | (33.3) | 3 | (20.0) | 2 | (13.3) | 10 | (66.7) | 4 | (80) | 20 | (37.7) |
| ADC-1 | | | | | | | | | | | | |

No TEAEs leading to death were related to the ADC-1 composition. The majority of hematological TEAEs were cytopenias and low grade gastrointestinal (GI) events and fatigue were commonly observed across all cohorts. Non-hematological grade 3≥TEAEs were infrequent and singular. Moreover, only 3 of the 17 serious adverse events (SAs) were related to the ADC-1 composition (on subject with leukopenia and neutropenia lasting>7 days at 3.0 mg/kg and one subject with thrombocytopenia at 3.5 mg/kg ADC-1). One case of interstitial lung disease (ILD) was observed in an SCLC subject at 3.0 mg/kg.

A summary of the efficacy of the ADC-1 composition in the dose escalation study is shown in Tables 4-7.

TABLE 4
Preliminary efficacy of the ADC-1 composition
in dose escalation (n = 48).

| | SCLC | NEN | Total |
|---|---|---|---|
| Response n (%) | (N = 23) | (N = 25) | (N = 48) |
| ORR | 14 (60.9%) | 7 (28.0%) | 21 (43.8%) |
| (confirmed CR/PR) | | | |
| CBR | 21 (91.3%) | 23 (92.0%) | 44 (91.7%) |
| (confirmed CR/PR + SD) | | | |

Note:
ORR = objective response rate; CBR = clinical benefit rate.

TABLE 5
Preliminary efficacy of the ADC-1 composition by dose in SCLC (n = 23).
Monotherapy ADC-1 Dose Escalation Response SCLC

| | 1.3 mg/kg | 1.8 mg/kg | 2.5 mg/kg | 3.0 mg/kg | 3.5 mg/kg | Total |
|---|---|---|---|---|---|---|
| | (N = 2) | (N = 7) | (N = 5) | (N = 7) | (N = 2) | (N = 23) |
| ORR (n(%)) | 1 (50.0) | 4 (57.1) | 2 (40.0) | 5 (71.4) | 2 (100) | 14 (60.9) |
| Confirmed | | | | | | |
| CR + PR | | | | | | |

TABLE 6
Preliminary efficacy of the ADC-1 composition by dose in NEN (n = 25).
Monotherapy ADC-1 Dose Escalation Response NEN

| | 1.3 mg/kg | 1.8 mg/kg | 2.5 mg/kg | 3.0 mg/kg | 3.5 mg/kg | Total |
|---|---|---|---|---|---|---|
| | (N = 0) | (N = 6) | (N = 9) | (N = 7) | (N = 1) | (N = 25) |
| ORR (n(%)) | 0 | 1 (12.5) | 3 (33.3) | 3 (42.9) | 0 | 7 (28.0) |
| Confirmed | | | | | | |
| CR + PR | | | | | | |

TABLE 7
Preliminary efficacy ADC-1 by dose in SCLC and NEN (n = 48).
Monotherapy ADC-1 Dose Escalation Response SCLC and NEN

| | 1.3 mg/kg | 1.8 mg/kg | 2.5 mg/kg | 3.0 mg/kg | 3.5 mg/kg | Total |
|---|---|---|---|---|---|---|
| | (N = 2) | (N = 15) | (N = 14) | (N = 14) | (N = 3) | (N = 48) |
| ORR (n(%)) | 1 (50.0) | 5 (33.3) | 5 (35.7) | 8 (57.1) | 2 (66.7) | 21 (43.8) |
| Confirmed | | | | | | |
| CR + PR | | | | | | |

Initial PK data indicates that the half-life of ADC-1 is about 7 days and the half-life of free Top1i is about 10 days across the studied doses.

### 8.3 Example 3: Dose Determination Results from a First-In-Human Study of ADC-1, a Novel SEZ6-Targeting Antibody-Drug Conjugate, in Advanced Solid Tumors

As of Aug. 27, 2024, 64 patients were enrolled with NENs. In the NEN cohort, patients had received a median of 3 (range 1-8) prior therapies. NEN histologies were large cell NEC (LCNEC; 22%, n=14), gastro-enteropancreatic NEC (GEPNEC; 19%, n=12), MTC (9%, n=6), neuroendocrine prostate carcinoma (NEPC; 8%, n=5), G3 NETs (8%, n=5), and other NECs (34%, n=22). The safety profile for ADC-1 was similar across NEN subtypes and aligned with the entire study population. The most frequent hematologic TEAEs were anemia (58%; G≥3: 45%), neutropenia (44%; G≥3: 33%), and thrombocytopenia (35%; G≥3: 21%). The most frequent nonhematologic TEAEs were fatigue (45%; G≥3: 3%) and nausea (38%; G≥3: 2%). The unadjudicated pneumonitis/interstitial lung disease rate was 4% (G≥3 in 2 pts). For the entire NEN cohort, the objective response rate (ORR) was 31.3% (20/64) and the clinical benefit rate was 92.2% (59/64). ORR by NEN type was: LCNEC, 28.6% (4/14); GEPNEC, 16.7% (2/12); NEPC, 60.0% (3/5); G3 NET, 60% (3/5); MTC, 16.7% (1/6); other NEC, 31.8% (7/22). The median duration of response was 5.59 months and median progression-free survival was 6.80 months.

### 8.4 Example 4: Dose Determination Results from a First-In-Human Study of ADC-1, a Novel SEZ6-Targeting Antibody-Drug Conjugate, in Advanced Solid Tumors

As of Jan. 3, 2025, the study has enrolled 206 subjects in all monotherapy cohorts that have received at least 1 dose of ADC-1, including 108 SCLC patients, 66 NEN patients, and 30 CNS patients. The following analysis is limited to subjects with at least 3 months follow up after first dose and includes 198 monotherapy subjects in the safety and PK analysis and 80 SCLC subjects in dose determination efficacy analysis.

The safety profile of ADC-1 is manageable and is shown in Table 8.

TABLE 8
Safety profile of the ADC-1 composition and all doses collected from clinical study as of Jan. 3, 2025.
Summary of Treatment Emergent Adverse Events (TEAEs): ADC-1 Monotherapy

| | 1.3 mg/kg | | 1.8 mg/kg | | 2.5 mg/kg | | 3.0 mg/kg | | 3.5 mg/kg | | Total | |
|---|---|---|---|---|---|---|---|---|---|---|---|---|
| | (n = 3) | | (n = 56) | | (n = 119) | | (n = 15) | | (n = 5) | | (n = 198) | |
| Any grade | 3 | (100) | 55 | (98.2) | 115 | (96.6) | 15 | (100) | 5 | (100) | 193 | (97.5) |
| TEAEs | | | | | | | | | | | | |
| Related to | 3 | (100) | 49 | (87.5) | 113 | (95.0) | 15 | (100) | 5 | (100) | 185 | (93.4) |
| ADC-1 | | | | | | | | | | | | |
| Grade ≥3 | 2 | (66.7) | 37 | (66.1) | 93 | (78.2) | 14 | (93.3) | 5 | (100) | 151 | (76.3) |
| TEAEs | | | | | | | | | | | | |
| Related to | 2 | (66.7) | 23 | (41.1) | 83 | (69.7) | 13 | (86.7) | 5 | (100) | 126 | (63.6) |
| ADC-1 | | | | | | | | | | | | |
| Serious | 1 | (33.3) | 28 | (50.0) | 47 | (39.5) | 6 | (40.0) | 2 | (40.0) | 84 | (42.4) |
| Adverse | | | | | | | | | | | | |
| Events | | | | | | | | | | | | |
| Related to | 0 | | 7 | (12.5) | 13 | (10.9) | 1 | (6.7) | 1 | (20.0) | 22 | (11.1) |
| ADC-1 | | | | | | | | | | | | |
| Dose | 0 | | 0 | | 0 | | 1 | (6.7) | 1 | (20.0) | 2 | (3.6) |
| Limiting | | | | | | | | | | | | |
| Toxicities | | | | | | | | | | | | |
| TEAE | 1 | (33.3) | 19 | (33.9) | 78 | (65.5) | 11 | (73.3) | 4 | (80.0) | 113 | (57.1) |
| Leading to | | | | | | | | | | | | |
| Dose | | | | | | | | | | | | |
| Interruption | | | | | | | | | | | | |
| Related to | 1 | (33.3) | 11 | (19.6) | 64 | (53.8) | 8 | (53.3) | 4 | (80.0) | 88 | (44.4) |
| ADC-1 | | | | | | | | | | | | |
| TEAE | 1 | (33.3) | 12 | (21.4) | 45 | (37.8) | 11 | (73.3) | 4 | (80.0) | 73 | (36.9) |
| Leading to | | | | | | | | | | | | |
| Dose | | | | | | | | | | | | |
| Reduction | | | | | | | | | | | | |

| | | | | | | | | | | | |
|---|---|---|---|---|---|---|---|---|---|---|---|
| Related to | 1 | (33.3) | 12 | (21.4) | 42 | (35.3) | 11 | (73.3) | 4 | (80.0) | 70 35.4) |

ADC-1

The most common TEAEs were hematologic toxicities (71.2%) which were dose dependent (higher incidence rate at 2.5 mg/kg (77.3%) versus 1.8 mg/kg (51.8%)), but were manageable with supportive care such as G-CSF and blood transfusions. The second most common TEAs were gastrointestinal (GI) toxicities (70.7%) with majority of the TEAs (94.8%) of grades 1 and 2. Of the GI toxicities, 92.5% did not require any dose modification. Of the grade≥3 TEAs, there was an observed higher incidence at 2.5 mg/kg (78.2%) versus the 1.8 mg/kg (66.1%).

A summary of the efficacy of the ADC-1 composition in the SCLC dose determination cohort is shown in Table 9.

TABLE 9
Efficacy of the ADC-1 composition SCLC dose determination
Monotherapy ADC-1 Dose Determination Response SCLC

| | |
|---|---|
| 1.8 mg/kg | 2.5 mg/kg |
| (N = 41) | (N = 39) |

Overall

| | | |
|---|---|---|
| ORR: Confirmed CR + | 23 (56.1) | 23 (59.0) |
| PR per INV, n (%) | | |
| ORR: Confirmed CR + | 26 (63.4) | 21 (53.8) |
| PR per ICR, n (%) | | |

Line of Therapy

| | | |
|---|---|---|
| 2L ORR: Confirmed | 13/16 (81.3) | 10/14 (71.4) |
| CR + PR, n/N (%) | | |
| 3L + ORR: Confirmed | 10/25 (40.0) | 13/25 (52.0) |
| CR + PR, n/N (%) | | |

Top1 Status

| | | |
|---|---|---|
| Naive ORR: Confirmed | 18/29 (62.1) | 18/28 (64.3) |
| CR + PR, n/N (%) | | |
| Exposed ORR: Confirmed | 5/12 (41.7) | 5/11 (45.5) |
| CR + PR, n/N (%) | | |

In the randomized dose determination cohorts, 56.1% and 59.0% of subjects had confirmed responses in the 1.8 and 2.5 mg/kg groups, respectively, based on investigator (INV) assessment. Based on a single independent reader's central review (ICR), the corresponding values were 63.4% (1.8 mg/kg) and 53.8% (2.5 mg/kg).

The median duration of response (DoR) in the dose determination cohort was 6.24 for the 1.8 mg/kg dose and 4.44 for the 2.5 mg/kg dose. The 1.8 and 2.5 mg/kg dose levels in dose determination cohort have a median PFS of 6.77 and 5.55 months, respectively.

### 8.5 Example 5: Phase 2/3 Study of the ADC-1 Composition in Combination with Atezolizumab Versus Platinum-Based Chemotherapy Plus Atezolizumab as First-Line Treatment in Subjects with Previously Untreated Extensive Stage Small Cell Lung Cancer (ES-SCLC

Disclosed herein is a Phase 2/3 study to evaluate optimal dose, safety, and efficacy of ADC-1 in combination with atezolizumab. Primary objectives are 1) to evaluate the safety and tolerability of ADC-1 in combination with atezolizumab, 2) determine and select the recommended Phase 3 dose (RP3D) of ADC-1 in combination with atezolizumab, 3) evaluate efficacy as measured by PFS of ADC-1 in combination with atezolizumab based on investigator assessment per RECIST Version 1.1, and 4) demonstrate the superiority of ADC-1 in combination with atezolizumab over standard of care in terms of efficacy as measured by OS.

Platinum-based chemotherapy has been the standard first-line treatment for several decades, the most common being platinum-etoposide combinations. Response rates to initial therapy (carboplatin or cisplatin in combination with a second agent, usually etoposide) are high, ranging from 70-90% for limited stage and 60-70% for extensive stage; however, recurrence rates are also high in the limited group and universal in the extensive group, leading to median survivals of 14-20 months and 9-11 months, respectively. The addition of atezolizumab or durvalumab to platinum-etoposide treatment has been approved for the first-line treatment of extensive-stage disease and demonstrated durable clinical activity and while maintaining a manageable safety profile in patients with ES-SCLC. Despite a 5 to 30% reduction in risk of death with the addition of immunotherapy, novel therapies are warranted.

The current first-line SCLC SOC is an anti-PD-L1 in combination with platinum-etoposide chemotherapy. The addition of atezolizumab to ADC-1 may extend the benefit CPI has demonstrated in combination with chemotherapy and provide a new therapeutic, platinum-etoposide chemotherapy-free option for the first-line treatment of ES-SCLC. This study will test the hypothesis that ADC-1 in combination with atezolizumab can be safely administered and extend clinical outcomes compared to standard of care in previously untreated ES-SCLC.

Up to 180 subjects will be enrolled in Phase 2 and approximately 550 subjects in Phase 3 with previously untreated ES-SCLC.

In Phase 2, subjects randomized to the investigational arms will receive ADC-1 at 1.5 mg/kg or 1.8 mg/kg Q3W IV in combination with atezolizumab. Subjects randomized to the control arm will receive carboplatin plus etoposide plus atezolizumab for 4 cycles, up to 6 cycles if tolerated, followed by atezolizumab maintenance. Subjects will receive study treatment until radiographic disease progression or meets other protocol defined discontinuation criteria.

In Phase 3, subjects randomized to the investigational arm will receive ADC-1 at the determined dose in combination with atezolizumab Q3W or carboplatin plus etoposide plus atezolizumab for 4 cycles, up to 6 if tolerated, followed by atezolizumab maintenance (SOC). Subjects randomized to ADC-1 in combination with atezolizumab or standard of care will receive study treatment until radiographic disease progression or meets other protocol defined discontinuation criteria.

### Key Eligibility Criteria:

Individuals at least 18 years old.
Diagnosis of histologically or cytologically confirmed ES-SCLC requiring treatment with first line therapy.
Subjects must have an Eastern Cooperative Oncology Group (ECOG) Performance Status of 0 to 1 during the screening period prior to the first dose of study treatment.
Subjects must have measurable disease per RECIST Version 1.1. Target lesions situated in a previously irradiated area are considered measurable if progression has been demonstrated in such lesions.
Subjects who have received chemoradiotherapy for consolidation therapy for previous LS-SCLC must be treated with curative intent and have a treatment-free interval of at least 6 months from the last course of platinum-based chemotherapy to the diagnosis of ES-SCLC and are no longer receiving CPI.
Subjects with brain metastasis from an extracranial solid tumor are eligible if the brain metastases are previously treated and stable without steroids or anticonvulsants within 7 days of the first dose of study treatment or untreated and asymptomatic without steroids or anticonvulsants within 7 days of the first dose of study treatment. Subjects with suspected brain metastases at screening should have a CT/MRI of the brain prior to study entry.
Subjects with known active/symptomatic CNS metastases should be excluded.
   Subjects with a history of interstitial lung disease (ILD) or pneumonitis that required treatment with systemic steroids, or any evidence of active ILD/pneumonitis on screening chest CT scan should be excluded.
Subjects should not have any clinically significant conditions that would adversely affect the subject's participation in the study major, and the subject should have a life expectancy of at least 3 months.

What is claimed:

1. A method of treating a small cell lung cancer (“SCLC”) tumor in a human subject in need thereof or population of human subjects in need thereof, comprising administering intravenously every three weeks to the human subject having said SCLC tumor a therapeutically effective amount of 1.3 mg/kg, 1.5 mg/kg, 1.8 mg/kg, 2.5 mg/kg, 3.0 mg/kg, or 3.5 mg/kg of an anti-SEZ6 antibody drug conjugate (“anti-SEZ6 ADC”) having the following structure: 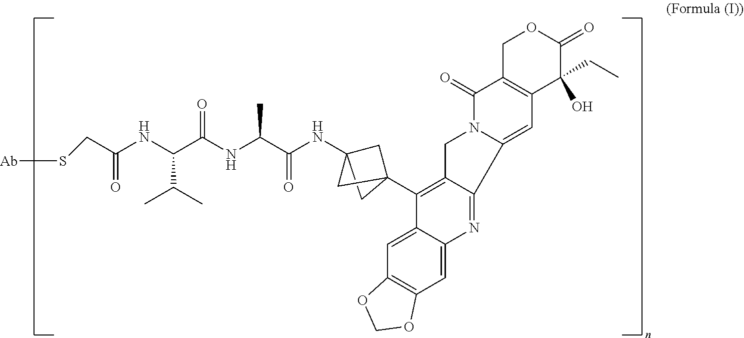
wherein n is an integer from 1 to 10, and wherein Ab is an anti-SEZ6 antibody comprising: two heavy chains each having an amino acid sequence shown as SEQ ID NO: 9 and two light chains each having an amino acid sequence shown as SEQ ID NO: 10, thereby treating said SCLC tumor.

2. The method of claim 1, wherein the SCLC tumor is refractory or relapsed.

3. The method of claim 1, wherein the SCLC tumor is previously untreated.

4-8. (canceled)

9. The method of claim 1, wherein the anti-SEZ6 ADC has an average drug-antibody ratio (DAR) of 6 or about 6.

10-11. (canceled)

12. The method of claim 1, wherein the SCLC tumor is a refractory or relapsed SCLC tumor, wherein a therapeutically effective amount of 1.8 mg/kg of the anti-SEZ6 ADC is administered intravenously every three weeks to the human subject, wherein the anti-SEZ6 ADC has an average DAR of about 6.

13-41. (canceled)

42. A method of treating a central nervous system (“CNS”) tumor in a human subject in need thereof or population of human subjects in need thereof, comprising administering intravenously every three weeks to the human subject having said CNS tumor a therapeutically effective amount of 1.3 mg/kg, 1.8 mg/kg, 2.5 mg/kg, 3.0 mg/kg, or 3.5 mg/kg of an anti-SEZ6 antibody drug conjugate (“anti-SEZ6 ADC”) having the following structure: 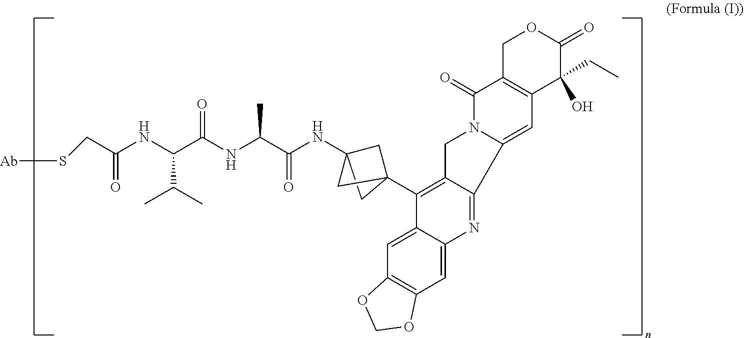
wherein n is an integer from 1 to 10, and wherein Ab is an anti-SEZ6 antibody comprising:
two heavy chains each having an amino acid sequence shown as SEQ ID NO: 9 and two light chains each having an amino acid sequence shown as SEQ ID NO: 10, thereby treating said CNS tumor.

43-62. (canceled)

63. A method of treating a high-grade neuroendocrine carcinoma/neuroendocrine tumor (“NEC/NET”) in a human subject in need thereof or population of human subjects in need thereof, comprising administering intravenously every three weeks to the human subject having said NEC/NET tumor a therapeutically effective amount of 1.3 mg/kg, 1.8 mg/kg, 2.5 mg/kg, 3.0 mg/kg, or 3.5 mg/kg of an anti-SEZ6 antibody drug conjugate (“anti-SEZ6 ADC”) having the following structure: 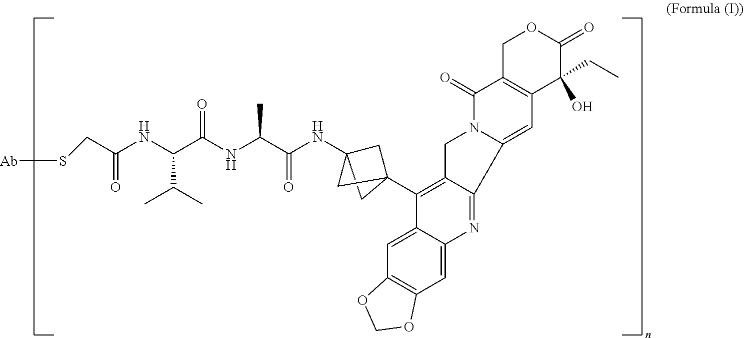
wherein n is an integer from 1 to 10, and wherein Ab is an anti-SEZ6 antibody comprising:
two heavy chains each having an amino acid sequence shown as SEQ ID NO: 9 and two light chains each having an amino acid sequence shown as SEQ ID NO: 10, thereby treating said NEC/NET tumor.

64-83. (canceled)

84. A method of treating a small cell lung cancer (“SCLC”) tumor in a human subject in need thereof or population of human subjects in need thereof, comprising administering intravenously every three weeks to the human subject having said SCLC tumor a therapeutically effective amount of 1.5 mg/kg or 1.8 mg/kg of an anti-SEZ6 antibody drug conjugate (“anti-SEZ6 ADC”) and an anti-PD-L1 antibody, the anti-SEZ6 ADC having the following structure: 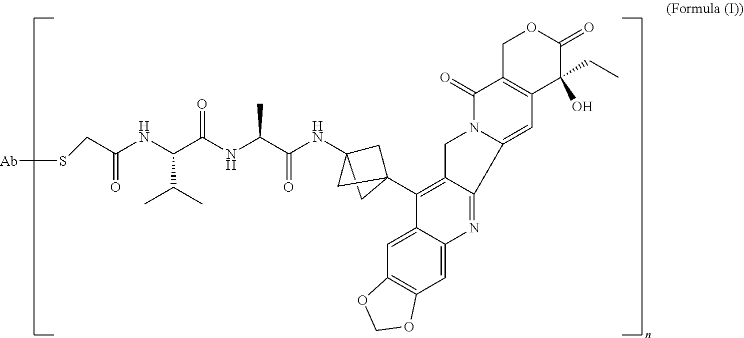
wherein n is an integer from 1 to 10, and wherein Ab is an anti-SEZ6 antibody comprising: two heavy chains each having an amino acid sequence shown as SEQ ID NO: 9 and two light chains each having an amino acid sequence shown as SEQ ID NO: 10, thereby treating said SCLC tumor.

85. The method of claim 84, wherein the anti-PD-L1 antibody is atezolizumab.


SEQUENCE LISTING (21 sequences)

SEQ ID NO: 1 - protein, 115 aa, synthetic construct
EVQLVQSGAEVKKPGESLKISCKGSGYSFTSSWINWVRQMPGKGLEWMGRIYPGEGDTNYNGNFEGQVTISADKSISTAYLQWSSLKASDTAMYYCTRGLVMDYWGQGTLVTVSS

SEQ ID NO: 2 - protein, 111 aa, synthetic construct
EIVLTQSPATLSLSPGERATLSCRASQSVDYNGISYMHWYQQKPGQAPRLLIYAASNVQSGIPARFSGSGSGTDFTLTISSLEPEDFAVYYCQQSIEDPPTFGGGTKVEIK

SEQ ID NO: 3 - protein, 10 aa, synthetic construct
GYSFTSSWIN

SEQ ID NO: 4 - protein, 17 aa, synthetic construct
RIYPGEGDTNYNGNFEG

SEQ ID NO: 5 - protein, 6 aa, synthetic construct
GLVMDY

SEQ ID NO: 6 - protein, 15 aa, synthetic construct
RASQSVDYNGISYMH

SEQ ID NO: 7 - protein, 7 aa, synthetic construct
AASNVQS

SEQ ID NO: 8 - protein, 9 aa, synthetic construct
QQSIEDPPT

SEQ ID NO: 9 - protein, 444 aa, synthetic construct
EVQLVQSGAEVKKPGESLKISCKGSGYSFTSSWINWVRQMPGKGLEWMGRIYPGEGDTNYNGNFEGQVTISADKSISTAYLQWSSLKASDTAMYYCTRGLVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 218 aa, synthetic construct
EIVLTQSPATLSLSPGERATLSCRASQSVDYNGISYMHWYQQKPGQAPRLLIYAASNVQSGIPARFSGSGSGTDFTLTISSLEPEDFAVYYCQQSIEDPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 10 aa, synthetic construct
GYTFTHYGMN

SEQ ID NO: 12 - protein, 17 aa, synthetic construct
WVNTYTGEPTYADDFKG

SEQ ID NO: 13 - protein, 9 aa, synthetic construct
EGEGLGFGD

SEQ ID NO: 14 - protein, 16 aa, synthetic construct
RSSQSIVHSHGDTYLE

SEQ ID NO: 15 - protein, 7 aa, synthetic construct
KVSNRFS

SEQ ID NO: 16 - protein, 9 aa, synthetic construct
FQGSHIPVT

SEQ ID NO: 17 - protein, 118 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSS

SEQ ID NO: 18 - protein, 113 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKR

SEQ ID NO: 19 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG